(19) United States
(12) Patent Application Publication
(10) Pub. No.: US 2025/0359925 A1
(43) Pub. Date: Nov. 27, 2025

(54) SYSTEM AND METHOD OF USING RF WIRE TO DISTINGUISH ANATOMY AFTER TRANSSEPTAL PUNCTURE

(71) Applicant: Boston Scientific Scimed, Inc., Maple Grove, MN (US)

(72) Inventor: Charlene Leung, Scarborough (CA)

(21) Appl. No.: 19/216,369

(22) Filed: May 22, 2025

Related U.S. Application Data
(60) Provisional application No. 63/651,124, filed on May 23, 2024.

(51) Int. Cl. A61B 18/14; A61B 18/00; A61B 18/12

(52) U.S. Cl. CPC A61B 18/1492; A61B 18/1206; A61B 2018/00184; A61B 2018/00351; A61B 2018/00666; A61B 2018/00839; A61B 2018/00898; A61B 2018/1475

(57) ABSTRACT

A transseptal surgical system to puncture an atrial septum within a heart includes an electrosurgical crossing assembly and a controller coupled to the crossing assembly. The crossing assembly includes a crossing member coupled to a radiofrequency (RF) energy source. The crossing member includes a crossing member distal tip having a crossing member electrode to deliver the RF energy. The controller can receive an intracardiac electrogram (EGM) reading signal from the crossing member electrode, compare the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy, and generate an alert if the crossing member electrode is determined to be within the aorta.

### CROSS REFERENCE TO RELATED APPLICATIONS

This application claims priority to U.S. Provisional Patent Application No. 63/651,124 entitled “METHOD OF USING RF WIRE TO DISTINGUISH ANATOMY AFTER TRANSSEPTAL PUNCTURE,” filed May 23, 2024, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical devices, assemblies, and systems that provide for a pressure sensing guidewire to puncture bodily tissues such as the atrial septum with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access percutaneously. Although the left atrium can be reached via the left ventricle and mitral valve, the catheter is manipulated through two U-turns, which can be cumbersome. The transseptal puncture is a technique of creating a small surgical passage through the atrial septum, or wall in the heart between the left and right atrium, through which a catheter can be fed. The atrial septum is punctured and dilated via tools to create the passage. The transseptal puncture permits a direct route to the left atrium via the atrial septum and systematic venous system. Increasing larger and complex medical devices can be passed into the left atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of guidewires having electrodes energized with a suitable power source such as an electrically coupled power generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to puncture tissue with plasma formed on the energized electrode. Tissue that contacts the plasma experiences a rapid vaporization of cellular fluid to produce a puncturing effect. Electrical energy can be applied to the electrodes either as a train of high frequency pulses or as a continuous signal typically in the radiofrequency (RF) range to perform the puncturing techniques.

### SUMMARY

In an Example 1, a transseptal surgical system configured to puncture an atrial septum within a heart of a patient, the transseptal surgical system comprising: an electrosurgical crossing assembly configured to couple to a radiofrequency (RF) energy source, the electrosurgical crossing assembly comprising: a delivery component having an elongate shaft defining a longitudinally extending lumen, and a crossing member adapted to be disposed within the lumen and coupled to the RF energy source, the crossing member having a crossing member distal tip extendable from the lumen, the crossing member distal tip having a crossing member electrode adapted to deliver the RF energy; and a controller coupled to the electrosurgical crossing assembly, the controller configured to: receive an intracardiac electrogram (EGM) reading signal from the crossing member electrode, compare the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy, and generate an alert if the crossing member electrode is determined to be within the aorta.

In an Example 2, the transseptal surgical system of Example 1, wherein the delivery component is a dilator having a tapered distal portion, the tapered distal portion including the delivery component distal tip.

In an Example 3, the transseptal surgical system of any of Examples 1 and 2, wherein the crossing member is a multifunction transseptal guidewire.

In an Example 4, the transseptal surgical system of any of Examples 1-3, wherein the crossing member electrode is a single electrode adapted to deliver the RF energy and receive the EGM reading signal.

In an Example 5, the transseptal surgical system of any of Examples 1-4, wherein the RF energy source is an RF electrosurgical generator.

In an Example 6, the transseptal surgical system of any of Examples 1-4, wherein the controller is incorporated into the RF electrosurgical generator.

In an Example 7, the transseptal surgical system of Example 6, wherein the RF electrosurgical generator includes a puncture setting adapted to provide RF energy to the crossing member and a sensing setting adapted to receive the EGM reading signal from the crossing member.

In an Example 8, the transseptal surgical system of any of Examples 1-5, wherein the controller is incorporated into an electroanatomical mapping (EAM) system.

In an Example 9, the transseptal surgical system of any of Examples 1-8, wherein the controller is configured to compare the EGM reading signal to both the aortic EGM characteristics and the left atrial EGM characteristics.

In an Example 10, the transseptal surgical system of any of Examples 1-9, wherein the controller is configured to compare the EGM reading to other EGM characteristics.

In an Example 11, the transseptal surgical system of any of Examples 1-10, wherein the aortic EGM characteristics and the left atrial EGM characteristics include information regarding amplitude and speed of a conduction profile.

In an Example 12, the transseptal surgical system of any of Examples 1-11, wherein the controller configured to generate the alert includes the controller configured to generate a visualization on a display device.

In an Example 13, the transseptal surgical system of any of Examples 1-12, wherein the controller is further configured to generate a confirmation notice if the crossing member electrode is determined to be within the left atrium.

In an Example 14, the transseptal surgical system of Example 13, wherein the confirmation notice includes an audio indication via a speaker.

In an Example 15, the transseptal surgical system of any of Examples 1-14, wherein the at least one of the aortic EGM characteristics and left atrial EGM characteristics includes thresholds and associated conductive profiles.

In an Example 16, a transseptal surgical system configured to puncture an atrial septum within a heart of a patient, the transseptal surgical system comprising: an electrosurgical crossing assembly configured to couple to a radiofrequency (RF) energy source, the electrosurgical crossing assembly comprising: a delivery component having an elongate shaft defining a longitudinally extending lumen, and a crossing member adapted to be disposed within the lumen and coupled to the RF energy source, the crossing member having a crossing member distal tip extendable from the lumen, the crossing member distal tip having a crossing member electrode adapted to deliver the RF energy; and a controller coupled to the electrosurgical crossing assembly, the controller configured to: receive an intracardiac electrogram (EGM) reading signal from the crossing member electrode, compare the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy, and generate an alert if the crossing member electrode is determined to be within the aorta.

In an Example 17, the transseptal surgical system of Example 16, wherein the delivery component is a dilator having a tapered distal portion, the tapered distal portion including the delivery component distal tip.

In an Example 18, the transseptal surgical system of Example 16, wherein the crossing member is a multifunction transseptal guidewire.

In an Example 19, the transseptal surgical system of Example 16, wherein the crossing member electrode is a single electrode adapted to deliver the RF energy and receive the EGM reading signal.

In an Example 20, the transseptal surgical system of Example 16, wherein the RF energy source is an RF electrosurgical generator.

In an Example 21, the transseptal surgical system of Example 16, wherein the controller is incorporated into the RF electrosurgical generator.

In an Example 22, the transseptal surgical system of Example 21, wherein the RF electrosurgical generator includes a puncture setting adapted to provide RF energy to the crossing member and a sensing setting adapted to receive the EGM reading signal from the crossing member.

In an Example 23, the transseptal surgical system of Example 16, wherein the controller is incorporated into an electroanatomical mapping (EAM) system.

In an Example 24, the transseptal surgical system of Example 16, wherein the controller is configured to compare the EGM reading signal to both the aortic EGM characteristics and the left atrial EGM characteristics.

In an Example 25, the transseptal surgical system of Example 16, wherein the controller is configured to compare the EGM reading to other EGM characteristics.

In an Example 26, the transseptal surgical system of Example 16, wherein the aortic EGM characteristics and the left atrial EGM characteristics include information regarding amplitude and speed of a conduction profile.

In an Example 27, the transseptal surgical system of Example 16, wherein the controller configured to generate the alert includes the controller configured to generate a visualization on a display device.

In an Example 28, the transseptal surgical system of Example 16, wherein the controller is further configured to generate a confirmation notice if the crossing member electrode is determined to be within the left atrium.

In an Example 29, the transseptal surgical system of Example 28, wherein the confirmation notice includes an audio indication via a speaker.

In an Example 30, the transseptal surgical system of Example 16, wherein the at least one of the aortic EGM characteristics and left atrial EGM characteristics includes thresholds and associated conductive profiles.

In an Example 31, a transseptal surgical system configured to puncture an atrial septum within a heart of a patient, the transseptal surgical system comprising: an electrosurgical crossing assembly configured to couple to a radiofrequency (RF) energy source, the electrosurgical crossing assembly comprising: a delivery component having an elongate shaft defining a longitudinally extending lumen, and a crossing member adapted to be disposed within the lumen and coupled to the RF energy source, the crossing member having a crossing member distal tip extendable from the lumen, the crossing member distal tip having a crossing member electrode adapted to deliver the RF energy; and a controller coupled to the electrosurgical crossing assembly, the controller configured to: receive an intracardiac electrogram (EGM) reading signal from the crossing member electrode, compare the EGM reading signal to both of aortic EGM characteristics having information to identify EGM signals emanating from the aorta and left atrial EGM characteristics having information to identify EGM signals emanating from the left atrium to determine a location of the crossing member electrode within cardiac anatomy, and generate one of an alert if the crossing member electrode is determined to be within the aorta and a confirmation notice if the crossing member electrode is determined to be within the left atrium.

In an Example 32, the transseptal surgical system of Example 31, wherein the at least one of the aortic EGM characteristics and left atrial EGM characteristics includes thresholds and associated conductive profiles.

In an Example 33, the transseptal surgical system of Example 31, wherein the alert and confirmation notice each include a visualization on a display device and an audio indication via a speaker.

In an Example 34, a method of puncturing an atrial septum with a heart of patient with a crossing member having a crossing member electrode adapted to deliver a radiofrequency (RF) energy, the method comprising: receiving an intracardiac electrogram (EGM) reading signal from the crossing member electrode; comparing the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy; and generating an alert if the crossing member electrode is determined to be within the aorta.

In an Example 35, the method of Example 34, and further comprising generating a confirmation notice if the crossing member electrode is determined to be within the left atrium.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator, a pressure monitor device, and a transseptal guidewire.

FIG. 2 is a block diagram illustrating an example process of the electrosurgical system of FIG. 1.

FIG. 3 is a schematic diagram illustrating an example controller configured for use with the electrosurgical system of FIG. 1 and the process of FIG. 2.

FIG. 4 is a block diagram illustrating an example process of the controller of FIG. 3.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

Transseptal punctures and other electrophysiology procedures are often performed with visualization and guidance systems to facilitate the delivery of catheters and other devices. The evolution of X-ray technologies and fluoroscopic guidance has enabled highly accurate imaging of complex anatomies, such imaging techniques, but expose patients and clinicians to ionizing radiation. Emerging non-fluoroscopic visualization technologies for electrophysiological procedures including transseptal punctures include three-dimensional electroanatomical mapping (EAM) and two-dimensional or three-dimensional intracardiac echocardiography (ICE) or transesophageal echocardiography (TEE). Certain regions of the world or clinicians, however, do not employ non-fluoroscopic visualization technologies.

Limited visualization of the transseptal apparatus on such techniques has been associated with reduced transseptal puncture success. For instance, transseptal punctures can inadvertently puncture the aorta, which sits anterior to the left atrium. Inadvertent puncture of the aorta with a relatively small device such as puncture electrode tip (in some examples, a puncture electrode is approximately 0.035 inches in outer diameter) does not present notable harm to a patient, but crossing a larger device such as a dilator (in some examples, a dilator is approximately 8.5F or 0.110″ in outer diameter) into the aorta creates a large hole in a relatively highly pressurized vessel that can lead to pericardial effusion and further surgical intervention.

Transseptal punctures and other electrophysiology procedures are routinely performed with inferential guidance systems such as pressure monitoring to confirm the location of punctures or chambers access in procedures. Pressure monitoring is typically a component of transseptal workflow and often relied upon for confirmation of access in procedures that do not employ non-fluoroscopic visualization technologies such as ICE. For example, a pressure reading is taken in the accessed location after a transseptal puncture to confirm left atrial access. Many clinicians do not use ultrasound modalities and simply use fluoroscopy and luminal pressure monitoring to confirm left atrial access.

In one example of transseptal puncture and pressure monitoring, an electrosurgical crossing member is removed from a delivery device after the interatrial septum has been punctured. The crossing member is replaced with a pressure sensing catheter that is threaded through the patient's vasculature to the puncture site. A pressure reading is taken with the pressure sensing catheter to confirm success of the transseptal puncture. The pressure sensing catheter can then be retracted and removed from the patient. Often, a guidewire is reinserted into the patient's vasculature to the puncture site to support the delivery of therapy devices to a therapy location in the heart. The multiple exchanges used to confirm location provides for inefficiencies in medical procedures. Multiple exchanges and vascular access of devices can also reduce efficacy and generate safety issues for both the patient and the clinicians.

Embodiments of the disclosed system provide for transseptal puncture and confirmation of the success of the transseptal puncture without device exchanges, pressure sensing, or ultrasound modalities. A transseptal guidewire having a puncture electrode is energized to puncture the atrial septum. Without removing the transseptal guidewire or the components of the access assembly, the system can determine an intracardiac electrogram (EGM) signal with the puncture electrode or different electrode on the transseptal guidewire. Based on the EGM signal, the system determines the location of the electrode, such as whether the electrode is in the aorta or the left atrium, which is used to inform whether to cross the puncture site with a larger device.

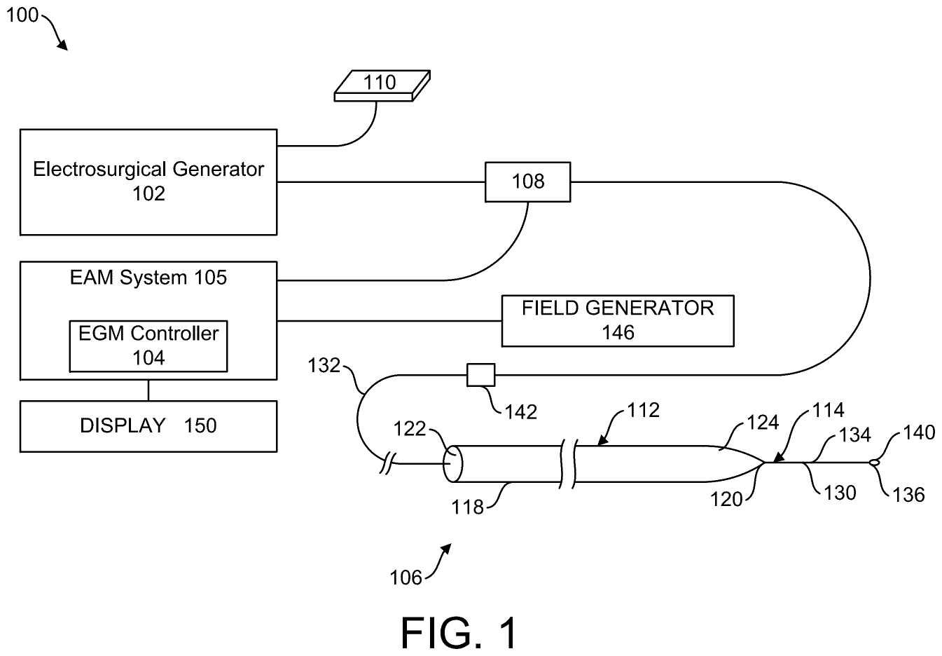

FIG. 1 illustrates an embodiment of an electrosurgical system 100 to facilitate vascular access to a heart and provide catheter positioning within cardiac anatomy. The embodiment of the electrosurgical system 100 includes an electrosurgical generator 102, an intracardiac electrogram (EGM) controller 104, and an electrosurgical crossing assembly 106. The EGM controller 104 can receive physiological signals from the heart provided by an electrode in the surgical assembly and detect and record EGM signals. The EGM controller 104 can be a standalone system or be incorporated into or included with other electrophysiological controllers into another electrophysiological system such as an electroanatomical mapping (EAM) system or into the electrosurgical generator 102. In the example illustrated electrosurgical system 100, the EGM controller 104 is incorporated into an EAM system 105. In the illustration, the electrosurgical crossing assembly 106 is electrically coupled to the electrosurgical generator 102 and the EAM system 104 via a multimode extension cable 108. The electrosurgical generator 102 is configured to provide a source of energy, such as radiofrequency (RF) energy to the electrosurgical crossing assembly 106 via the cable 108. In some embodiments, the electrosurgical system 100 includes a ground pad electrode, or indifferent (dispersive) patch electrode 110 electrically coupled to the generator 102 for use with the electrosurgical crossing assembly 106 in a monopolar configuration. In some embodiments, the electrosurgical assembly 106 is implemented in a bipolar configuration without an indifferent patch electrode.

The electrosurgical crossing assembly 106 of the illustrated embodiment includes a delivery component 112 and a crossing member that, in one embodiment, is configured as a transseptal guidewire 114. The delivery component 112 includes an elongated shaft 118 having a shaft distal tip 120. The elongated shaft 118 defines a longitudinally extending axial lumen 122. The transseptal guidewire 114 is adapted to be disposed within the lumen 122 and coupled to the RF energy source, such as the generator 102. In some embodiments, the delivery component 112 can include an elongate sheath, and the transseptal guidewire 114 is disposed within the sheath. In another embodiment, the delivery component 112 can include a dilator/sheath assembly, and the transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE).

The transseptal guidewire 114 includes a puncture wire shaft 130 with a puncture wire proximal portion 132 and a puncture wire distal portion 134 having a puncture wire distal tip 136. The puncture wire distal tip 136 includes a distal tip electrode 140 adapted to deliver the RF energy and detect physiological signals in the heart. The puncture wire proximal portion 132 includes an end connector 142 configured to electrically couple to cable 108. The transseptal guidewire 108 is configured to conduct RF energy from the proximal portion 132 along the puncture wire shaft 130 to the electrode 140 and to conduct detected electrical physiological signals from the electrode 140 along the puncture wire shaft 130 to the proximal portion 132. In some embodiments, the puncture wire shaft 130 is constructed from an electrically conductive material having an insulative outer coating, and the distal tip electrode 140 is exposed. In some embodiment, the electrically conductive material is a flexible, shape memory material such as a nickel titanium alloy or nitinol. In the illustrated embodiment, the distal tip electrode 140 is selectively electrically couplable via the multimode extension cable 108 to the RF generator 102 and the EAM system 105. In some embodiments, the electrosurgical assembly 106 also includes a plurality of tracking electrodes (not shown) that are electrically coupled to the EAM system 105 such as via cable 108.

In the illustrated example, the transseptal guidewire 114 is configured as a multifunction conductive guidewire. For instance, the transseptal guidewire 114 can be used, without exchanges, as a guidewire, a transseptal puncture device, a sensing electrode, and as an exchange rail for delivering therapy sheaths. Such embodiments provide efficiencies to medical procedures as the transseptal guidewire 114 performs multiple functions and reduces the amount of device exchanges in the medical procedure. The transseptal guidewire 114 includes the distal tip 136 extendable from the delivery component shaft distal tip 120 such that the delivery component 112 is retractable from the patient over the guidewire 114 with the guidewire distal tip 136 disposed within the heart. The transseptal guidewire 114 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 136, the distal portion 134 is biased to form a coil for anchoring the transseptal guidewire 114 beyond the puncture. The delivery component 112 is retractable from the patient over the transseptal guidewire 114 with the distal tip 136 still disposed within the heart. The transseptal guidewire 114 can also support the installation of therapy devices to a therapy location in the patient's heart, such as tubular members or other catheters and for advancing other devices within the heart.

The electrosurgical generator 102 is configured to provide the source of RF energy to the electrosurgical assembly 106 for a puncture operation. The generator 102 is electrically coupled via cables to the electrosurgical assembly 106 and patch electrode 110. During a monopolar punction operation of electrosurgical generator 102, a first electrode, often referred to as the active electrode, is provided with the electrosurgical crossing assembly 106 such as distal tip electrode 140 on the transseptal guidewire 114 while another electrode, such as patch electrode 110, is typically located on the back, buttocks, upper leg, or other suitable anatomical location of the patient during surgery and remote from the active electrode. In such a configuration, the patch electrode 110 is often referred to as a patient return electrode. RF energy for a puncture function in a monopolar mode may be provided at a selected voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical tools in addition to the transseptal guidewire 114. In one example, the electrosurgical generator 102 provides RF energy to the active electrode as an alternating current having a frequency in the range of 100 kHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected transseptal guidewire 114. The actual pulses are often sinusoidal or square waves and bi-phasic, that is alternating positive and negative amplitudes.

The cable 108, which can include a switch, can be employed to configure the transseptal guidewire 114 in a plurality of settings. For example, the cable 108 can be configured in a puncture setting to electrically couple the electrosurgical generator 102 with the transseptal guidewire 114 via cable 108. In the puncture setting, the electrosurgical generator 102 provides the RF energy to an active electrode on the transseptal guidewire 114 via the cable 108. An electrical circuit of RF energy is formed between the active electrode 140 and the patch electrode 110 through the patient, which is used to puncture tissue at the active electrode. The cable 108 can also be configured in a mapping or sensing setting to electrically couple the electrosurgical assembly 106 with the EGM controller 104 via cable. In the mapping or sensing setting, an electrode on the transseptal guidewire 114 provides detected physiological signals to the EAM system 105, such as the EGM controller 104. In the illustrated embodiment, the distal tip electrode 140 can operate as an active electrode in the puncture setting and also provide physiological signals, such as EGM signals from the heart, in the mapping or sensing setting via selective operation of the cable 108. In other embodiments, a dedicated puncture electrode on the transseptal guidewire 114 is configurable as the active electrode and a dedicated sensing electrode on the transseptal guidewire 114 is configurable detect and provide physiological signals via selective operation of the cable 108. In still another embodiment, the switch to select the puncture and mapping or sensing settings can be incorporated into the electrosurgical assembly 106. In an example in which the EGM controller 104 is incorporated into the electrosurgical generator 102, the switch to select the puncture and sensing settings can be included in the circuitry of the electrosurgical generator 102.

The EAM system 105 can also include a tracking controller that is operable to track the locations of the various elements of the electrosurgical assembly 106 such as with respect to the structures of the heart or with respect to each other. The illustrated EAM system 105 includes a localization field generator 146, a mapping and navigation controller (not shown), and a display device 150. The EAM system 105 generates high-fidelity three-dimensional anatomical and electro-anatomical maps of the cardiac chambers of interest. In one embodiment, the cable 108 can be configured to the mapping or sensing setting to electrically couple the tracking component of the EAM system 105 with the electrosurgical assembly 106 and allow the EAM system 105 to track the location of the electrosurgical assembly 106. The system 100 can be implemented in conjunction with an additional visualization system such as a fluoroscopic visualization system employing a C-arm, or an echocardiogram system such as an intracardiac echocardiography (ICE) system a transesophageal electrocardiography (TEE) system (not shown).

The EAM system 105 is operable to track the location of the various components of the electrosurgical device 106, and to generate high-fidelity three-dimensional anatomical and electro-anatomical maps of the heart, including portions of the heart such as cardiac chambers of interest or other structures of interest such as the sinoatrial node or atrioventricular node as visualizations on display 150. The EAM system 105 includes one or more controllers, such as microprocessors or computers, that execute code out of memory to control or perform functional aspects of the EAM system 105 including the EGM controller 104 and tracking controller and mapping and navigation controller.

The EAM system 105 generates a localization field via the field generator 126 to define a localization volume about the heart. The cable 108 can be switched to a mapping setting, and a location sensor or sensing element on a tracked device, such as electrodes on the electrosurgical assembly 106, generate an output that is provided to the EAM system 105. The signals from the electrosurgical assembly 106 are processed by the mapping and navigation controller to track the location of the electrodes, and consequently, the corresponding components of the electrosurgical assembly 106, within the localization volume. In one embodiment, impedance tracking methodologies are be employed to track the locations of the electrosurgical assembly 106. For instance, the localization field is an electric field generated, for example, by an external field generator arrangement of the field generator 146, such as surface electrodes, and is received by intra-body or intra-cardiac devices, such as an intracardiac catheter, or both. In these examples, the location sensing elements can constitute electrodes on the tracked devices that generate outputs received and processed by the mapping and navigation controller 148 to track the location of the various location sensing electrodes within the localization volume. In another embodiment, the device tracking is accomplished using magnetic tracking techniques, in which the field generator is a magnetic field generator that generates a magnetic field defining the localization volume, and location sensors on the tracked devices are magnetic field sensors. The EAM system 105 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor.

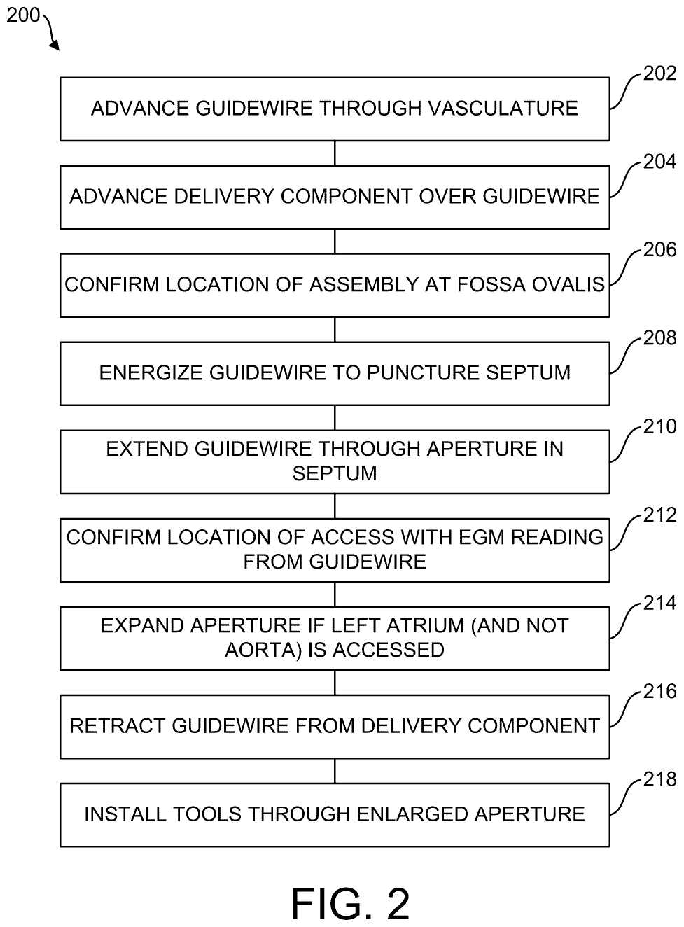

FIG. 2 illustrates an example method 200 of an anticipated use of the system 100. The electrosurgical assembly 106 is coupled to the RF generator 102 and EGM controller 104 via cable 108. If the electrosurgical assembly 106 is to be configured in a monopolar mode, the patch electrode 110 is coupled to the patient. The RF generator 102 can be set to a puncture mode, such as an energy output of approximately 50 watts. In some examples, femoral access is obtained via a conventional percutaneous needle, and the transseptal guidewire 114 is inserted into the vasculature and advanced to the superior vena cava at 202. The shaft distal tip 120 of the delivery component 116 is advanced over the proximal portion 132 of the guidewire 114, and the distal tapered portion 124 of the delivery component shaft 118 is advanced over the guidewire 114 to the superior vena cava at 204. Under visualization, the distal tapered portion 124 is moved from the superior vena cava to the right atrial septum and then to the fossa ovalis of the heart. The location of the assembly 106 is confirmed at the fossa ovalis at 206. For example, the delivery component distal tip 120 is confirmed at the fossa ovalis at 206, such as via visualization, and the electrode 140 of the transseptal guidewire 114 is advanced from the delivery component distal tip 120. In another example, the electrode 140 can be used to confirm the position of the transseptal guidewire 114 at the fossa ovalis at 206. For instance, the cable 108 (or electrosurgical assembly 106) is configured in a mapping or sensing setting to electrically couple the electrode 140 with the EGM controller 104. An EGM reading can be taken with the electrode 140 to determine the location of the distal portion 134 of the transseptal guidewire 114 based on EGM characteristics of the EGM reading, and whether the EGM characteristics conform with the fossa ovalis instead of the rest of the right atrial tissue.

The transseptal guidewire 114 is energized to puncture the fossa ovalis at 208. The cable 108 (or electrosurgical assembly 106) is configured in a puncture setting to electrically couple the RF generator 102 with the electrode 140. In one example, the exposed electrode 140 of the transseptal guidewire 114 is extended a few millimeters from the delivery component distal tip 120 to tent the heart tissue, and the transseptal guidewire 114 can be locked in position with respect to the delivery component 112. Forward pressure is applied to the electrosurgical device 106 and the transseptal guidewire 114 is actuated to apply the RF energy to the electrode 140 and puncture the fossa ovalis at 208. The RF energy punctures the fossa ovalis and creates an aperture in the fossa ovalis. The transseptal guidewire 114 is unlocked from the delivery component 112, and the transseptal guidewire 114 is extended through the aperture in the fossa ovalis at 210. In general, the transseptal guidewire 108 is extended longitudinally for several millimeters prior to the distal portion 134 curving to assume a J-tip or pigtail shape and deflecting away from the atrial septum.

The transseptal guidewire 114 is employed to confirm left atrial access with an EGM signal at 212. Without removing the transseptal guidewire 108 or the components of the access assembly, such as the delivery component, the system 100 can be configured to determine an EGM signal at the access location. The cable 108 (or electrosurgical assembly 106) is configured in a mapping or sensing setting to electrically couple the electrode 140 with the EGM controller 104. The transseptal guidewire 114 is advanced through the aperture in the atrial septum, and an EGM reading is taken with the electrode 140 to determine the location of the distal portion 134 of the transseptal guidewire 114. Prior to enlarging the aperture, such as enlarging the aperture via crossing a dilator, the EAM system 105 can confirm access into the left atrium based on EGM characteristics taken from the EGM reading. If confirmed, the transseptal guidewire 114 can be advanced into the left atrium of the heart through the aperture and anchored.

In the embodiment of the delivery component 116 configured as the dilator/sheath assembly, the distal tapered portion 124 is advanced into the puncture site to expand the aperture 214. The delivery component 112 can be retracted from the patient over the transseptal guidewire 114 at 216, and transseptal guidewire 114 can provide support the installation of tubular members or other catheters and for advancing other devices within the heart through the enlarged or expanded aperture at 218.

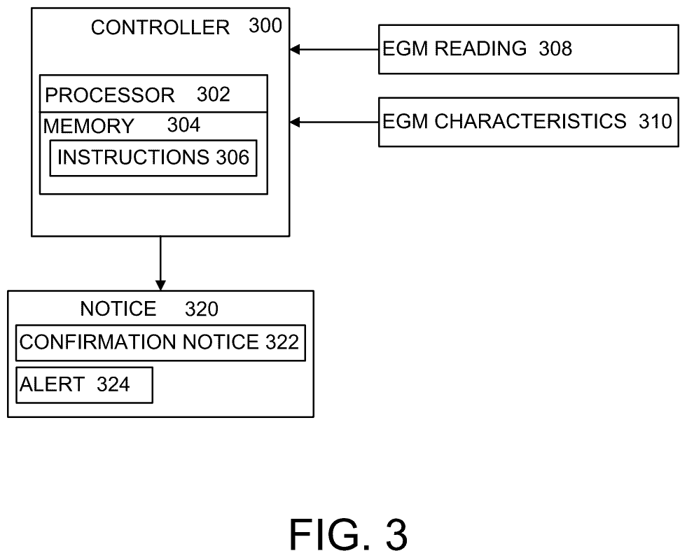

FIG. 3 illustrates an example controller 300 that can be implemented with EAM system 104 in the electrosurgical system 100 such as via EGM controller 104. In one embodiment, controller 300 can be implemented with the EAM system 105 or as part of an electrophysiological recording system. In still another embodiment, the controller 300 can be implemented into a generator, such as electrosurgical generator 102. In this example, the electrosurgical generator 102 can incorporate circuitry to switch between the puncture setting and the mapping and sensing setting, or, more particularly, a puncture setting and an electrode location detector setting to determine the location of the electrode based on a received EGM reading. In such an example electrosurgical generator 102, the cable 108 can still be used such as with the EAM system 105, or the cable 108 (and EAM system) can be omitted from electrosurgical system 100 for a transseptal crossing procedure.

The controller 300 is electrically couplable to the electrode 140 of the transseptal guidewire 114 such as via the mapping and sensing setting of the cable 108 (or electrosurgical assembly 106). The controller 300 is implemented to receive an EGM signal from the electrode 140 and determine whether the EGM signal is indicative of an EGM signal from the left atrium or an EGM from the aorta to confirm left atrial access of the transseptal guidewire 114. The controller 300 generates a notice, such as a confirmation notification as an audio indication or a visualization if the EGM signal received from the electrode 140 compares favorably with an EGM signal expected from the left atrium, or an alert as an alarming audio indication or a visualization if the EGM signal received from the electrode 140 compares favorably with an EGM signal expected from the aorta. The controller 300 can include a processor 302 and a memory 304. The memory 304 stores processor executable instructions 306. For instance, the processor executable instructions 306 can be in the form of a program, such as a computer program or application. The processor 302 is implemented to execute the instructions 306 that configure the controller 300. In some embodiments, the controller 300 is implemented as a computing device such as a laptop computer, a workstation, a desktop computer, a tablet, or a smartphone. In some embodiments, the controller 300 includes additional components such as a display, a touchscreen, speakers or other output devices, a keyboard or other input devices, or communication circuitry such as computer network adapters.

In some embodiments, the processor 302 includes a plurality of main processing cores to run an operating system and perform general-purpose tasks on an integrated circuit. The processor 302 can also include built-in logic or a programmable functional unit, also on the same integrated circuit with an instruction-set architecture. In additional to multiple general-purpose, main processing cores and the application processing unit, the controller 300 in embodiments includes other devices or circuits such as graphics processing units or neural network processing units, which may include heterogeneous or homogenous instruction set architectures with the main processing cores.

Memory 304 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 302. Any such computer storage media may be part of the controller 300 and implemented as memory 304. Memory 304 is a non-transitory, processor readable memory device. Accordingly, a propagating signal by itself does not qualify as storage media or memory 304.

The controller 300 is configured to receive inputs or information from the electrosurgical system 100, such as an EGM reading signal 308 as an electrical signal input from the electrosurgical assembly 106 and computer files of an EGM characteristic 310, for storage in memory 304 and use by the instructions 306. The EGM reading signal 308 is an electrical signal detected by the electrosurgical assembly 106 after puncture of the atrial septum to determine the location of the distal portion 134 of the transseptal guidewire 114. In one example, the EGM characteristic 310 can include information to identify a source location of the EGM reading signal 308 in the cardiac anatomy. In examples, the EGM characteristic can include a plurality of EGM characteristics that includes information to identify a set of source locations of EGM reading signals 308 in various locations of the cardiac anatomy. For instance, EGM characteristics can include aortic EGM characteristics that includes information to identify EGM reading signals 308 emanating from the aorta and can include left atrial EGM characteristics that includes information to identify EGM reading signals 308 emanating from the left atrium. Such information can include thresholds, a conductive profile, and other identifying information for EGM signals emanating from the aorta and the left atrium. In one example, the left atrium includes a conduction profile of the associated contractile cardiac tissue that is distinguishable in amplitude and speed from a conduction profile of associated non-contractile and smooth muscle tissue in the aorta. Based on the conductive profile of an EGM signals taken from the aorta, for example, first thresholds can be assigned to speed and amplitude above and below a first set of selected amounts such that an EGM signal having speed and amplitude within the thresholds are identified as emanating from the aorta. Also, based on conductive profile of an EGM signals taken from the left atrium, for example, second thresholds can be assigned to speed and amplitude above and below a second set of selected amounts such that an EGM reading having speed and amplitude within the thresholds are identified as emanating from the left atrium. In examples, computer files for the EGM characteristics 310 can also include fossa ovalis EGM characteristics that includes information for identifying EGM signals emanating from the fossa ovalis and other right atrial septum EGM characteristics that includes information for identifying EGM signals emanating from the right atrial septum other than the fossa ovalis.

The controller 300 is configured to generate a notice 320 of whether the EGM reading signal is associated with an EGM characteristic from the aorta or the left atrium. The notice 320 can be presented on an output device such as a speaker or a display 130 of the EAM system 104. In the illustrated embodiment, the notice 320 can be part of a visualization or audio tone that can notify a user of whether the EGM reading signal 308 compares with an EGM characteristic 310 of an EGM signal from the left atrium or the aorta. For instance, the notice 320 can include a confirmation notice 322, such as a green light with a pleasant audio signal to indicate to the user that the left atrium has been accessed, and the notice can include an alert 324, such as a red, flashing light with an alarming audio signal to indicate to the user that aorta has been accessed.

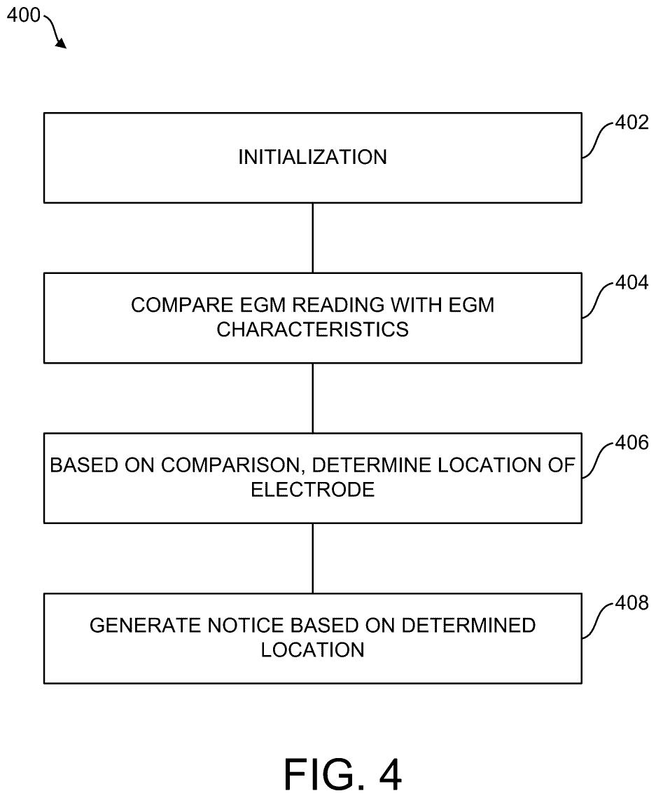

FIG. 4 illustrates a process 400 of configuring the controller 300 to receive an EGM reading signal from the electrode 104 of a transseptal guidewire 114 and determine whether the EGM reading signal is indicative of an EGM signal from the left atrium or an EGM signal from the aorta to confirm left atrial access of the transseptal guidewire 114. In embodiments, the process 400 is a computerized process. In one embodiment, process 400 is implemented as set of processor-executable instructions, such as instructions 306, stored in a non-transitory memory, such as memory 304 to be executed by a processor 302 to configure controller 300. The instructions 306 to implement process 400 can be configured to receive information, such as to retrieve from memory 304 an EGM reading 308 and EGM characteristics 310. Further, the instructions 306 to implement process 400 can be configured to generate an alert such as an audio alarm or a visualization, such as visualization 320 on a display of graphical representations on display device 130.

The controller is initialized at 402. For example, the EGM characteristics 310 are loaded into memory 304 for use by instructions 306. In one instance, the EGM characteristics 310 loaded into memory 304 for use by instructions 306 can include aortic EGM characteristics that includes information to identify EGM signals emanating from the aorta and can include left atrial EGM characteristics that includes information to identify EGM signals emanating from the left atrium. In some embodiments, the controller 300 can automatically configure the electrosurgical assembly 106 to receive an EGM reading, such as to couple the electrode 140 to measurement circuitry rather than to RF puncture circuitry. In some embodiments, a user can configure the electrosurgical assembly 106 to receive the EGM reading via a switch on cable 108 (or electrosurgical assembly 106), but this feature can be configured via the controller 300 in several embodiments. In some embodiments, the process 400 applies a success flag, which is reset (or not set) as part of initialization at 402.

The process 400 compares the EGM reading signal 308 with the EGM characteristics to determine a location type of the EGM reading signal 308 at 404. In one embodiment, the process 400 compares the EGM reading signal 308 to at least one of aortic EGM characteristics having information to identify EGM signals emanating from the aorta and left atrial EGM characteristics having information to identify EGM signals emanating from the left atrium. For example, the EGM reading signal 308 is compared to one of the aortic EGM characteristics and the left atrial EGM characteristics to determine whether the EGM reading signal compares favorably with the compared-to EGM characteristic. In another example, the EGM reading signal 308 is compared to both of the aortic EGM characteristics and the left atrial EGM characteristics to determine which of the EGM characteristics compares favorably to the EGM reading signal 308 or whether the EGM reading signal compares unfavorably to at least one of the EGM characteristics. In still another example, the EGM reading signal 308 is compared to the aortic EGM characteristics, the left atrial EGM characteristics, and other locational EGM characteristics to determine which of the EGM characteristics compares favorably to the EGM reading signal 308 or whether the EGM reading signal compares unfavorably to at least one of the EGM characteristics.

Based on whether the EGM reading signal compares favorably or unfavorably to the aortic EGM characteristics and the left atrial EGM characteristics, the location of the electrode 140 within the cardiac anatomy can be determined at 406. Further, if the EGM reading signal compares favorably with one of the aortic EGM characteristics and the left atrial EGM characteristics and unfavorably with the other of the left atrial characteristics and the aortic EGM characteristics, a success flag can be set at 406. To begin the process, the success flag is not set. If the EGM reading signal compares favorably to the aortic EGM characteristics and unfavorably to the left atrial EGM characteristics, process 400 at 406 determines the location of the electrode 140 providing the EGM reading signal is in the aorta and the success flag is set. If, however, the EGM reading signal compares unfavorably to the aortic EGM characteristics and favorably to the left atrial EGM characteristics, process 400 at 406 determines the location of the electrode 140 providing the EGM reading signal is in the left atrium and the success flag is set. Still, if the EGM reading signal compares favorably to the aortic EGM characteristics and favorably to the left atrial EGM characteristics, process 400 at 406 determines an error has occurred and can restart process 400. The success flag is not set. If the EGM reading signal compares unfavorably to the aortic EGM characteristics and unfavorably to the left atrial EGM characteristics, process 400 at 406 can compare the EGM reading signal to other EGM characteristics prior to determining an error has occurred.

The process 400 generates a notice if a location of the electrode 140 is determined, such as if the success flag is set, at 408. In one example, the notice at 408 is a confirmation notification as a pleasant audio indication via speakers or a visualization on a graphical display if the location of the electrode 104 is determined to be in the left atrium at 406. Also, the notice at 408 is an alert such as an alarming audio indication with the speakers or a visualization on the graphical display if the location of the electrode 104 is determined to be in the aorta at 406. In some embodiments, the process 400 is capable of generating both a confirmation notification and the alert as notice at 408 depending on the corresponding location determined at 406.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A transseptal surgical system configured to puncture an atrial septum within a heart of a patient, the transseptal surgical system comprising:
an electrosurgical crossing assembly configured to couple to a radiofrequency (RF) energy source, the electrosurgical crossing assembly comprising:
a delivery component having an elongate shaft defining a longitudinally extending lumen, and
a crossing member adapted to be disposed within the lumen and coupled to the RF energy source, the crossing member having a crossing member distal tip extendable from the lumen, the crossing member distal tip having a crossing member electrode adapted to deliver the RF energy; and
a controller coupled to the electrosurgical crossing assembly, the controller configured to:
receive an intracardiac electrogram (EGM) reading signal from the crossing member electrode,
compare the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy, and
generate an alert if the crossing member electrode is determined to be within the aorta.

2. The transseptal surgical system of claim 1, wherein the delivery component is a dilator having a tapered distal portion, the tapered distal portion including the delivery component distal tip.

3. The transseptal surgical system of claim 1, wherein the crossing member is a multifunction transseptal guidewire.

4. The transseptal surgical system of claim 1, wherein the crossing member electrode is a single electrode adapted to deliver the RF energy and receive the EGM reading signal.

5. The transseptal surgical system of claim 1, wherein the RF energy source is an RF electrosurgical generator.

6. The transseptal surgical system of claim 1, wherein the controller is incorporated into the RF electrosurgical generator.

7. The transseptal surgical system of claim 6, wherein the RF electrosurgical generator includes a puncture setting adapted to provide RF energy to the crossing member and a sensing setting adapted to receive the EGM reading signal from the crossing member.

8. The transseptal surgical system of claim 1, wherein the controller is incorporated into an electroanatomical mapping (EAM) system.

9. The transseptal surgical system of claim 1, wherein the controller is configured to compare the EGM reading signal to both the aortic EGM characteristics and the left atrial EGM characteristics.

10. The transseptal surgical system of claim 1, wherein the controller is configured to compare the EGM reading to other EGM characteristics.

11. The transseptal surgical system of claim 1, wherein the aortic EGM characteristics and the left atrial EGM characteristics include information regarding amplitude and speed of a conduction profile.

12. The transseptal surgical system of claim 1, wherein the controller configured to generate the alert includes the controller configured to generate a visualization on a display device.

13. The transseptal surgical system of claim 1, wherein the controller is further configured to generate a confirmation notice if the crossing member electrode is determined to be within the left atrium.

14. The transseptal surgical system of claim 13, wherein the confirmation notice includes an audio indication via a speaker.

15. The transseptal surgical system of claim 1, wherein the at least one of the aortic EGM characteristics and left atrial EGM characteristics includes thresholds and associated conductive profiles.

16. A transseptal surgical system configured to puncture an atrial septum within a heart of a patient, the transseptal surgical system comprising:
an electrosurgical crossing assembly configured to couple to a radiofrequency (RF) energy source, the electrosurgical crossing assembly comprising:
a delivery component having an elongate shaft defining a longitudinally extending lumen, and
a crossing member adapted to be disposed within the lumen and coupled to the RF energy source, the crossing member having a crossing member distal tip extendable from the lumen, the crossing member distal tip having a crossing member electrode adapted to deliver the RF energy; and
a controller coupled to the electrosurgical crossing assembly, the controller configured to:
receive an intracardiac electrogram (EGM) reading signal from the crossing member electrode,
compare the EGM reading signal to both of aortic EGM characteristics having information to identify EGM signals emanating from the aorta and left atrial EGM characteristics having information to identify EGM signals emanating from the left atrium to determine a location of the crossing member electrode within cardiac anatomy, and
generate one of an alert if the crossing member electrode is determined to be within the aorta and a confirmation notice if the crossing member electrode is determined to be within the left atrium.

17. The transseptal surgical system of claim 16, wherein the at least one of the aortic EGM characteristics and left atrial EGM characteristics includes thresholds and associated conductive profiles.

18. The transseptal surgical system of claim 16, wherein the alert and confirmation notice each include a visualization on a display device and an audio indication via a speaker.

19. A method of puncturing an atrial septum with a heart of patient with a crossing member having a crossing member electrode adapted to deliver a radiofrequency (RF) energy, the method comprising:
receiving an intracardiac electrogram (EGM) reading signal from the crossing member electrode;
comparing the EGM reading signal to at least one of aortic EGM characteristics having information to identify EGM signals emanating from the aorta and left atrial EGM characteristics having information to identify EGM signals emanating from the left atrium to determine a location of the crossing member electrode within cardiac anatomy; and
generating an alert if the crossing member electrode is determined to be within the aorta.

20. The method of claim 19, and further comprising generating a confirmation notice if the crossing member electrode is determined to be within the left atrium.
